(19) United States
(12) Patent Application Publication
(10) Pub. No.: US 2011/0166357 A1
(43) Pub. Date: Jul. 7, 2011

(54) METHOD FOR PRODUCING N-SUBSTITUTED-TRANS-4-AZIDOPIPERIDINE-3-OL

(75) Inventors: Osamu Tokuda, Ibaraki (JP); Toshiaki Aikawa, Osaka (JP); Tetsuya Ikemoto, Osaka (JP)

(73) Assignee: SUMITOMO CHEMICAL COMPANY, LIMITED, Chuo-ku, Tokyo (JP)

(21) Appl. No.: 13/062,967

(22) PCT Filed: Sep. 2, 2009
(86) PCT No.: PCT/JP2009/065623

§ 371 Date: Mar. 9, 2011

(30) Foreign Application Priority Data
Sep. 12, 2008 (JP) 2008-234472

(51) Int. Cl. C07D 211/98

(52) U.S. Cl. 546/242

(57) ABSTRACT

An N-substituted-trans-4-azidopiperidin-3-ol represented by formula (II-1)

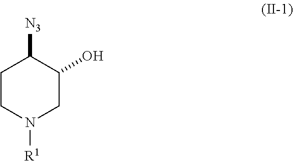

R¹ is as defined below, which is useful as a pharmaceutical intermediate and so on, is produced by reacting an N-substituted-3,4-epoxypiperidine represented by formula (I):

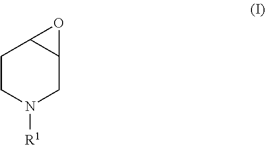

wherein R¹ represents an aralkyl group having 7 to 24 carbon atoms or an alkyl group having 1 to 12 carbon atoms, with sodium azide in the presence of an inorganic lithium salt.

### TECHNICAL FIELD

The present invention relates to a method for producing an N-substituted-trans-4-azidopiperidin-3-ol.

### BACKGROUND ART

A method for producing an N-protected-trans-4-azidopiperidin-3-ol by reacting 3,4-epoxypiperidine, in which a nitrogen atom on a piperidine ring is protected, with sodium azide is described in J. Med. Chem. 41, 3563-3567 (1998). However, according to this method, there arises a problem that position selectivity of introduction of an azido group is poor and a large amount of an N-protected-trans-3-azidopiperidine-4-ol is produced as a by-product.

### DISCLOSURE OF THE INVENTION

In the present invention, an N-substituted-trans-4-azidopiperidin-3-ol is selectively obtained by reacting an N-substituted-3,4-epoxypiperidine with sodium azide in the presence of an inorganic lithium salt. An N-substituted-trans-4-azidopiperidin-3-ol is obtained by reducing an azido group of the product and, furthermore, an intermediate for the production of a useful pharmaceutical described in WO2007/039462 or the like can be obtained by removing a substituent on a nitrogen atom constituting a piperidine ring.

That is, the present invention provides a method for producing an N-substituted-trans-4-azidopiperidin-3-ol represented by formula (II-1):

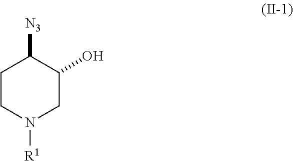

wherein R¹ is as defined below, by reacting an N-substituted-3,4-epoxypiperidine represented by formula (I):

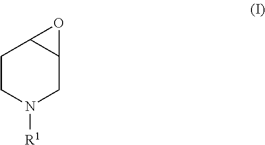

wherein R¹ represents an alkyl group having 1 to 12 carbon atoms or an aralkyl group having 7 to 24 carbon atoms, with sodium azide in the presence of an inorganic lithium salt.

Further, the present invention provides a method for producing an amino compound represented by formula (III-A):

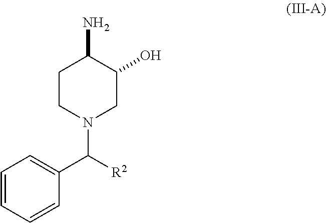

wherein R² is as defined below, by reducing an azide compound represented by formula (II-A):

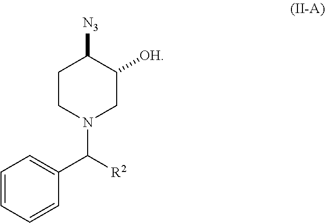

wherein R² represents an aralkyl group having 7 to 17 carbon atoms, an alkyl group having 1 to 11 carbon atoms, a phenyl group or a hydrogen atom.

Furthermore, the present invention provides a method for protecting an amino group of an amino compound represented by formula (III-A) to obtain a carbamate compound represented by formula (IV-A):

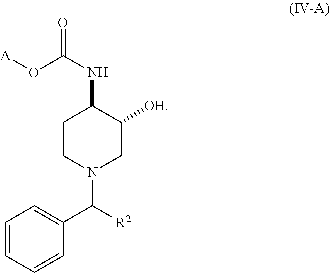

wherein R² is as defined above, and A represents an alkyl group having 1 to 12 carbon atoms; and then producing a trans-4-alkoxycarbonylaminopiperidin-3-ol represented by formula (V-A):

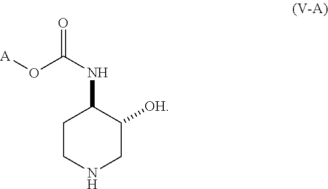

wherein A is as defined above.

According to the present invention, an N-substituted-trans-4-azidopiperidin-3-ol represented by formula (II-1), which is useful as a pharmaceutical intermediate, is selectively obtained. Therefore, since a step of resolving position isomers after the reaction is not required, the present invention is industrially beneficial. Further, the product can be converted to an amino compound by reducing the azido group.

Examples of the alkyl group having 1 to 12 carbon atoms represented by R¹ in formula (I) include a methyl group, an ethyl group, a propyl group, an isopropyl group, a butyl group, an isobutyl group, a tert-butyl group, a pentyl group, a hexyl group, a heptyl group, an octyl group, a nonyl group, a decyl group, an undecyl group, and a dodecyl group. An aralkyl group having 7 to 24 carbon atoms is a group having one or more aromatic hydrocarbon atoms such as a phenyl group and a naphthyl group on these alkyl groups having 1 to 12 carbon atoms, and examples thereof include a benzyl group, a 1-phenylethyl group, a 2-phenylethyl group, a 1-naphthylethyl group, a 1-phenylpropyl group, a 2-phenylpropyl group, a 3-phenylpropyl group, a 1-phenyl-1-methylethyl group, a 1-phenylbutyl group, a 2-phenylbutyl group, a 3-phenylbutyl group, a 4-phenylbutyl group, a 1-phenyl-1-methylpropyl group, and a diphenylmethyl group. R¹ is preferably an aralkyl group having 7 to 24 carbon atoms from the viewpoint of easiness of elimination and, for example, it is more preferably an aralkyl group in which the 1-position of an alkyl group is substitute with a phenyl group, such as a benzyl group or a 1-phenylethyl group, and particularly preferably a benzyl group.

Examples of the N-substituted-3,4-epoxypiperidine represented by formula (I) (hereinafter abbreviated to Compound (I)) include 3-methyl-7-oxa-3-azabicyclo[4.1.0]heptane, 3-ethyl-7-oxa-3-azabicyclo[4.1.0]heptane, 3-benzyl-7-oxa-3-azabicyclo[4.1.0]heptane, 3-(1-phenylethyl)-7-oxa-3-azabicyclo[4.1.0]heptane, 3-(2-phenylethyl)-7-oxa-3-azabicyclo[4.1.0]heptane, 3-propyl-7-oxa-3-azabicyclo[4.1.0]heptane, 3-isopropyl-7-oxa-3-azabicyclo[4.1.0]heptane, 3-butyl-7-oxa-3-azabicyclo[4.1.0]heptane, 3-(1-phenylpropyl)-7-oxa-3-azabicyclo[4.1.0]heptane, 3-(2-phenylpropyl)-7-oxa-3-azabicyclo[4.1.0]heptane, 3-(3-phenylpropyl)-7-oxa-3-azabicyclo[4.1.0]heptane, 3-(1-phenyl-1-methylethyl)-7-oxa-3-azabicyclo[4.1.0]heptane, 3-(1,1-diphenylmethyl)-7-oxa-3-azabicyclo[4.1.0]heptane, 3-butyl-7-oxa-3-azabicyclo[4.1.0]heptane, and 3-isobutyl-7-oxa-3-azabicyclo[4.1.0]heptane. Compound (I) may be a racemic form or an optically active form. Compound (I) is preferably a compound capable of easily removing a substituent R¹, for example, 3-benzyl-7-oxa-3-azabicyclo[4.1.0]heptane, 3-(1-phenylethyl)-7-oxa-3-azabicyclo[4.1.0]heptane or the like, and particularly preferably 3-benzyl-7-oxa-3-azabicyclo[4.1.0]heptane. Compound (I) can be produced, for example, in accordance with a known method described in Chem. Pharm. Bull., 29, 3026 (1981) or the like.

Examples of the inorganic lithium salt include lithium chloride, lithium bromide, lithium iodide, lithium perchlorate, lithium periodate, lithium carbonate, lithium sulfate, and lithium phosphate. Among these inorganic lithium salts, lithium halides such as lithium chloride, lithium bromide and lithium iodide, and lithium perhalogenates such as lithium perchlorate and lithium periodate are preferable. Lithium chloride and lithium perchlorate are more preferable. It is also possible to use, as the inorganic lithium salt, a commercially available inorganic lithium salt, and those prepared by any known method.

It is also possible to use, as sodium azide, commercially available sodium azide, and those prepared by any known method.

In the reaction of Compound (I) with sodium azide in the presence of an inorganic lithium salt, the use amount of the inorganic lithium salt is usually from 0.1 to 10 mol, and preferably from 1 to 5 mol, based on 1 mol of Compound (I). Also, the use amount of sodium azide is usually from 1 to 3 mol, and preferably from 1 to 2 mol, based on 1 mol of Compound (I).

This reaction is usually carried out in a solvent. The solvent may be inert to the reaction, and examples thereof include aliphatic hydrocarbon solvents such as pentane, hexane, isohexane, heptane, isoheptane, octane, isooctane, nonane, isononane, decane, isodecane, undecane, dodecane, cyclopentane, cyclohexane, methylcyclohexane, tert-butylcyclohexane and petroleum ether; aromatic solvents such as benzene, toluene, ethylbenzene, isopropylbenzene, tert-butylbenzene, xylene, mesitylene, monochlorobenzene, monofluorobenzene, α,α,α-trifluoromethylbenzene, 1,2-dichlorobenzene, 1,3-dichlorobenzene, 1,2,3-trichlorobenzene and 1,2,4-trichlorobenzene; ether solvents such as tetrahydrofuran, methyltetrahydrofuran, diethyl ether, dipropyl ether, diisopropyl ether, dibutyl ether, dipentyl ether, dihexyl ether, diheptyl ether, dioctyl ether, tert-butyl methyl ether, cyclopentyl methyl ether, 1,2-dimethoxyethane, diethylene glycol dimethyl ether, anisole and diphenyl ether; alcohol solvents such as methanol, ethanol, 1-propanol, 2-propanol, butyl alcohol, isobutyl alcohol, tert-butyl alcohol, 1-pentanol, 2-pentanol, isopentyl alcohol, 1-hexanol, 2-hexanol, isohexyl alcohol, 1-heptanol, 2-heptanol, 3-heptanol, isoheptyl alcohol, ethylene glycol monomethyl ether, ethylene glycol monoethyl ether, ethylene glycol monopropyl ether, ethylene glycol monoisopropyl ether, ethylene glycol monobutyl ether, ethylene glycol monoisobutyl ether, ethylene glycol mono-tert-butyl ether, diethylene glycol monomethyl ether, diethylene glycol monoethyl ether, diethylene glycol monopropyl ether, diethylene glycol monoisopropyl ether, diethylene glycol monobutyl ether, diethylene glycol monoisobutyl ether and diethylene glycol mono-tert-butyl ether; ester solvents such as ethyl acetate, propyl acetate, isopropyl acetate, butyl acetate, isobutyl acetate, tert-butyl acetate, amyl acetate and isoamyl acetate; nitrile solvents such as acetonitrile and propionitrile; aprotic polar solvents such as dimethyl sulfoxide, sulfolane, N,N-dimethylformamide, N,N-dimethylacetamide, N,N-dimethylpropionamide, N-methylpyrrolidone, γ-butyrolactone, dimethyl carbonate, diethyl carbonate, ethylene carbonate, propylene carbonate, 1,3-dimethyl-2-imidazolidinone and 1,3-dimethyl-3,4,5,6-tetrahydro-2(1H)-pyridinone; and water. These solvents may be used alone, or a mixture thereof may be used. Nitrile solvents are preferable and, among these nitrile solvents, acetonitrile is more preferable. The use amount of the solvent is usually from 1 to 50 L, and preferably from 2 to 15 L, based on 1 kg of Compound (I).

The reaction temperature is usually from 0 to 100° C., and preferably from 40 to 80° C. The reaction time varies depending on the reaction temperature, the reaction reagent, the use amount of the solvent and the like, and usually is from 1 to 10 hours. Proceeding of the reaction can be confirmed by conventional means such as thin-layer chromatography, gas chromatography, high-performance liquid chromatography.

There is no particular limitation on the order of mixing reaction reagents. For example, mixing can be carried out by a method of adding sodium azide and an inorganic lithium salt in any order to Compound (I) or a solution thereof.

An N-substituted-trans-4-azidopiperidin-3-ol represented by formula (II-1) (hereinafter abbreviated to Compound (II-1)) is contained as a main product in the mixture obtained after completion of the reaction.

Although an N-substituted-trans-3-azidopiperidine-4-ol represented by formula (II-2):

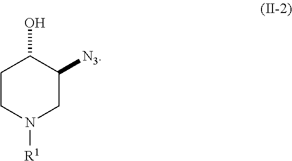

wherein R¹ is as defined above (hereinafter abbreviated to Compound (II-2)) is sometimes contained as a by-product, a production ratio of them is usually within a range from Compound (II-1):Compound (II-2)=95:5 to 100:0

When the mixture containing Compound (II-1) obtained after completion of the reaction is, for example, subjected to conventional post-treatments such as filtration, extraction and washing with water and then subjected to conventional isolation treatments such as distillation, crystallization, Compound (II-1) can be obtained alone, or as a mixture with Compound (II-2). At this time, Compound (II-1) may be isolated as a salt of any acid such as hydrochloric acid, benzoic acid or tartaric acid. The thus obtained Compound (II-1) or a salt thereof can be further purified by conventional purification treatments such as recrystallization; extraction and purification; distillation; treatment of adsorption to activated carbon, silica, alumina and the like; and chromatography such as silica gel column chromatography.

Examples of Compound (II-1) include trans-4-azido-1-methylpiperidin-3-ol, trans-4-azido-1-ethylpiperidin-3-ol, trans-4-azido-1-benzylpiperidin-3-ol, trans-4-azido-1-propylpiperidin-3-ol, trans-4-azido-1-isopropylpiperidin-3-ol, trans-4-azido-1-(1-phenylethyl)piperidin-3-ol, trans-4-azido-1-(2-phenylethyl)piperidin-3-ol, trans-4-azido-1-(1,1-diphenylmethyl)piperidin-3-ol, trans-4-azido-1-butylpiperidin-3-ol, trans-4-azido-1-(1-phenylpropyl)piperidin-3-ol, trans-4-azido-1-(2-phenylpropyl)piperidin-3-ol, trans-4-azido-1-(3-phenylpropyl)piperidin-3-ol, and trans-4-azido-1-(1-phenyl-2-methylethyl)piperidin-3-ol. When a racemic form is used as Compound (I), the obtained Compound (II-1) is also usually a racemic form and, when an optically active form is used as Compound (I), the obtained Compound (II-1) is also usually an optically active form. Also, a trans form of Compound (II-1) means one in which each of an azido group and a hydroxyl group is on opposite sides of the piperidine ring. Although the compound in which each of an azido group and a hydroxyl group is on the same side of the piperidine ring is a cis form, a cis form is not usually produced in the present invention.

In the present reaction, when a compound represented by formula (I-A):

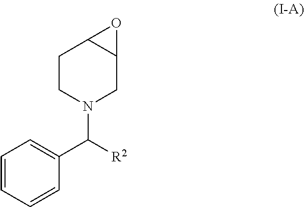

wherein R² is as defined above (hereinafter abbreviated to Compound (I-A)) is used as Compound (I), an azide compound represented by formula (II-A) (hereinafter abbreviated to Compound (II-A)) is obtained as Compound (II-1).

Examples of the alkyl group having 1 to 11 carbon atoms represented by R² in formula (I-A) include a methyl group, an ethyl group, a propyl group, an isopropyl group, a butyl group, an isobutyl group, a tert-butyl group, a pentyl group, a hexyl group, a heptyl group, an octyl group, a nonyl group, a decyl group, an undecyl group. The aralkyl group having 7 to 17 carbon atoms is a group having one or more aromatic hydrocarbon groups such as a phenyl group and a naphthyl group on these alkyl groups having 1 to 11 carbon atoms, and examples thereof include a benzyl group, a 1-phenylethyl group, a 2-phenylethyl group, a 1-naphthylethyl group, a 1-phenylpropyl group, a 2-phenylpropyl group, a 3-phenylpropyl group, a 1-phenyl-1-methylethyl group, a 1-phenylbutyl group, a 2-phenylbutyl group, a 3-phenylbutyl group, a 4-phenylbutyl group, and a 1-phenyl-1-methylpropyl group. R² is preferably a hydrogen atom.

Examples of Compound (I-A) include 3-benzyl-7-oxa-3-azabicyclo[4.1.0]heptane, 3-(1-phenylethyl)-7-oxa-3-azabicyclo[4.1.0]heptane, 3-(1-phenylpropyl)-7-oxa-3-azabicyclo[4.1.0]heptane, 3-(1-phenylbutyl)-7-oxa-3-azabicyclo[4.1.0]heptane, 3-(1-phenyl-2-methylpropyl)-7-oxa-3-azabicyclo[4.1.0]heptane, and 3-(1,3-diphenylpropyl)-7-oxa-3-azabicyclo[4.1.0]heptane. Among these compounds, 3-benzyl-7-oxa-3-azabicyclo[4.1.0]heptane is preferable. Also, Compound (I-A) may be a racemic form, or an optically active form.

Examples of Compound (II-A) include trans-4-azido-1-benzylpiperidin-3-ol, trans-4-azido-1-(1-phenylethyl)piperidin-3-ol, trans-4-azido-1-(1-phenylpropyl)piperidin-3-ol, trans-4-azido-1-(1-phenylbutyl)piperidin-3-ol, trans-4-azido-1-(1-phenyl-2-methylpropyl)piperidin-3-ol, and trans-4-azido-1-(1,3-diphenylpropyl)piperidin-3-ol. When a racemic form is used as Compound (I-A), the obtained Compound (II-A) is also usually a racemic form and, when an optically active form is used as Compound (I-A), the obtained Compound (II-A) is also usually an optically active form.

The method for producing an amino compound represented by formula (III-A) (hereinafter abbreviated to Compound (III-A)) by reducing Compound (II-A) will be described in more detail below. In this method, an azido group of Compound (II-A) is converted into an amino group by reduction.

As Compound (II-A), the above mixture obtained after completion of the reaction may be used as it is, or used after a post-treatment. Further, the isolated Compound (II-A) or a salt thereof may be used, and further purified Compound (II-A) or a salt thereof may be used.

Reduction of the azido group is carried out by reacting Compound (II-A) with a conventional reducing agent. Examples of the reducing agent include hydrogen, a metal halide (for example, lithium aluminum hydride), and a phosphine compound (for example, triphenylphosphine). Reduction by hydrogen is carried out, for example, in the presence of palladium on carbon (the palladium on carbon may contain sulfur). In particular, the reaction of Compound (II-A) with hydrogen in the presence of sulfur-containing palladium on carbon is preferable. This hydrogenation will be described below.

The sulfur-containing palladium on carbon may be a wet product or a dry product. The content of a palladium atom in palladium on carbon is usually from 0.5 to 50% by weight, and preferably from 5 to 15% by weight, and the content of a sulfur atom is usually from 0.01 to 1% by weight, and preferably from 0.05 to 0.2% by weight. It is also possible to use, as the sulfur-containing palladium on carbon, commercially available sulfur-containing palladium, and those prepared by any known method. The use amount of the sulfur-containing palladium on carbon is usually within a range from 0.1 to 50 g, and preferably from 1 to 20 g, in terms of the palladium atom based on 1 kg of Compound (II-A). Palladium supported on carbon is usually zero-valent and when a di- or tetra-valent palladium compound is supported, it is preferably used after reducing to zero valence by a conventional method.

It is also possible to use, as hydrogen, a commercially available hydrogen gas, and those generated by any known method. The hydrogen pressure during the reaction is usually from 0.05 to 5 MPa, and preferably from 0.1 to 0.5 MPa. It is also possible to use as a mixed gas with an inert gas such as nitrogen or argon. In that case, the hydrogen pressure during the reaction is the same as the above hydrogen pressure.

Hydrogenation is usually carried out in a solvent. The solvent which is inert to the reaction is available, and examples thereof include aliphatic hydrocarbon solvents such as pentane, hexane, isohexane, heptane, isoheptane, octane, isooctane, nonane, isononane, decane, isodecane, undecane, dodecane, cyclopentane, cyclohexane, methylcyclohexane, tert-butylcyclohexane and petroleum ether; ether solvents such as tetrahydrofuran, methyltetrahydrofuran, diethyl ether, dipropyl ether, diisopropyl ether, dibutyl ether, dipentyl ether, dihexyl ether, diheptyl ether, dioctyl ether, tert-butyl methyl ether, cyclopentyl methyl ether, 1,2-dimethoxyethane and diethylene glycol dimethyl ether; alcohol solvents such as methanol, ethanol, 1-propanol, 2-propanol, butyl alcohol, isobutyl alcohol, tert-butyl alcohol, 1-pentanol, 2-pentanol, isopentyl alcohol, 1-hexanol, 2-hexanol, isohexyl alcohol, 1-heptanol, 2-heptanol, 3-heptanol, isoheptyl alcohol, ethylene glycol monomethyl ether, ethylene glycol monoethyl ether, ethylene glycol mono-n-propyl ether, ethylene glycol monoisopropyl ether, ethylene glycol monobutyl ether, ethylene glycol monoisobutyl ether, ethylene glycol mono-tert-butyl ether, diethylene glycol monomethyl ether, diethylene glycol monoethyl ether, diethylene glycol monopropyl ether, diethylene glycol monoisopropyl ether, diethylene glycol monobutyl ether, diethylene glycol monoisobutyl ether and diethylene glycol mono-tert-butyl ether; ester solvents such as ethyl acetate, propyl acetate, isopropyl acetate, butyl acetate, isobutyl acetate, tert-butyl acetate, amyl acetate and isoamyl acetate; aprotic polar solvents such as dimethyl sulfoxide, sulfolane, N,N-dimethylformamide, N,N-dimethylacetamide, N,N-dimethylpropionamide, N-methylpyrrolidone, γ-butyrolactone, dimethyl carbonate, diethyl carbonate, ethylene carbonate, propylene carbonate, 1,3-dimethyl-2-imidazolidinone and 1,3-dimethyl-3,4,5,6-tetrahydro-2(1H)-pyridinone; and water. These solvents may be used alone, or a mixture thereof may be used. Alcohol solvents are preferable and, among these alcohol solvents, ethanol is preferable. The use amount of the solvent is usually from 1 to 50 L, and preferably from 2 to 15 L, based on 1 kg of Compound (II-A).

The reaction temperature is usually from −20 to 80° C., and preferably from 0 to 40° C. The reaction time varies depending on the reaction temperature, the reaction reagent, the use amount of the solvent, the hydrogen pressure and the like, and is usually from 1 to 5 hours. Proceeding of the reaction can be confirmed by conventional means such as thin-layer chromatography, gas chromatography, high-performance liquid chromatography.

There is no particular limitation on the order of mixing of reaction reagents. For example, mixing can be carried out by a method of mixing Compound (II-A) or a solution thereof with sulfur-containing palladium on carbon and adding hydrogen to the obtained mixture, a method for adding Compound (II-A) to sulfur-containing palladium on carbon under a hydrogen atmosphere and the like. A method for mixing a solution of Compound (II-A) with sulfur-containing palladium on carbon and adding hydrogen to the obtained mixture is preferable.

The mixture obtained after completion of the reaction contains Compound (III-A) and, for example, when such mixture is subjected to conventional post-treatments such as filtration, extraction, washing with water, and then subjected to conventional isolation treatments such as distillation, crystallization, Compound (III-A) can be obtained. At this time, Compound (III-A) may be isolated as a salt of any acid such as hydrochloric acid, benzoic acid or tartaric acid. The thus isolated Compound (III-A) or a salt thereof can be further purified by conventional purification treatments such as recrystallization; extraction and purification; distillation; treatments of absorption to activated carbon, silica, alumina and the like; and chromatography such as silica gel column chromatography.

Examples of Compound (III-A) include trans-4-amino-1-benzylpiperidin-3-ol, trans-4-amino-1-(1-phenylethyl)piperidin-3-ol, trans-4-amino-1-(1-phenylpropyl)piperidin-3-ol, trans-4-amino-1-(1-phenylbutyl)piperidin-3-ol, trans-4-amino-1-(1-phenyl-2-methylpropyl)piperidin-3-ol, and trans-4-amino-1-(1,3-diphenylpropyl)piperidin-3-ol, and trans-4-amino-1-benzylpiperidin-3-ol is preferable. When a racemic form is used as Compound (II-A), the obtained Compound (III-A) is also usually a racemic form and, when an optically active form is used as Compound (II-A), the obtained Compound (III-A) is also usually an optically active form.

The step of protecting an amino group of Compound (III-A) to obtain a compound represented by the above formula (IV-A) (hereinafter abbreviated to Compound (IV-A)), and then deprotecting the compound to obtain a trans-4-protected aminopiperidin-3-ol compound represented by the above formula (V-A) (hereinafter abbreviated to Compound (V-A)) will be described below.

As Compound (III-A) to be subjected to the step of protecting an amino group, the above mixture containing the same obtained after completion of the reaction may be used as it is, or used after the above post-treatment. As a matter of course, the isolated Compound (III-A) or a salt thereof may be used, or further purified Compound (III-A) or a salt thereof may be used.

Examples of the alkyl group having 1 to 12 carbon atoms represented by A in formula (IV-A) include a methyl group, an ethyl group, a propyl group, an isopropyl group, a butyl group, an isobutyl group, a tert-butyl group, a pentyl group, a hexyl group, a heptyl group, an octyl group, a nonyl group, a decyl group, an undecyl group, and a dodecyl group. An ethyl group, an isopropyl group and a tert-butyl group are preferable, and a tert-butyl group is more preferable.

An amino group is protected by converting Compound (III-A) to Compound (IV-A). Protection is usually carried out by reacting Compound (III-A) in the presence of an alkyl halocarbonate or a dialkyl carbonate and a base. Herein, the alkyl halocarbonate is represented by the formula (VI-1):

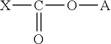

wherein X represents a halogen atom such as a chlorine atom or a bromine atom, and A is as defined above, and the dialkyl carbonate is represented by formula (VI-2):

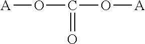

wherein A is as defined above.

Examples of the base include alkali metal hydroxides such as potassium hydroxide, sodium hydroxide and lithium hydroxide; alkali metal carbonates such as potassium carbonate, sodium carbonate and lithium carbonate; tertiary amine compounds such as triethylamine and diisopropylethylamine; alkali metal alkoxides such as sodium methoxide, sodium ethoxide, sodium tert-butoxide and potassium tert-butoxide; alkali metal hydrides such as sodium hydride and potassium hydride; alkali earth metal hydrides such as calcium hydride; alkyl metal compounds such as n-butyl lithium; and alkali metal amide compounds such as lithium diisopropylamide and lithium hexamethyldisilazide. Among these bases, tertiary amine compounds are preferable.

Examples of the alkyl halocarbonate include methyl chlorocarbonate, ethyl chlorocarbonate, propyl chlorocarbonate and butyl chlorocarbonate. Examples of the dialkyl carbonate include di-tert-butyl carbonate. In order to convert Compound (III-A) to a carbamate compound, it is preferred to react dialkyl carbonate, and particularly preferably di-tert-butyl carbonate.

The use amount of the base is usually from 1 to 10 mol, and preferably from 1 to 3 mol, based on 1 mol of Compound (III-A). The use amount of the alkyl halocarbonate or dialkyl carbonate is usually from 1 to 5 mol, and preferably from 1 to 2 mol, based on 1 mol of Compound (III-A). It is also possible to use, as these reagents, a commercially available reagent, and those prepared by a known method.

Protection with an amino group is usually carried out in a solvent. The solvent which is inert to the reaction is available, and examples thereof include aliphatic hydrocarbon solvents such as pentane, hexane, isohexane, heptane, isoheptane, octane, isooctane, nonane, isononane, decane, isodecane, undecane, dodecane, cyclopentane, cyclohexane, methylcyclohexane, tert-butylcyclohexane and petroleum ether; aromatic solvents such as benzene, toluene, ethylbenzene, isopropylbenzene, tert-butylbenzene, xylene, mesitylene, monochlorobenzene, monofluorobenzene, α,α,α-trifluoromethylbenzene, 1,2-dichlorobenzene, 1,3-dichlorobenzene, 1,2,3-trichlorobenzene and 1,2,4-trichlorobenzene; ether solvents such as tetrahydrofuran, methyltetrahydrofuran, diethyl ether, dipropyl ether, diisopropyl ether, dibutyl ether, dipentyl ether, dihexyl ether, diheptyl ether, dioctyl ether, tert-butyl methyl ether, cyclopentyl methyl ether, 1,2-dimethoxyethane, diethylene glycol dimethyl ether, anisole and diphenyl ether; aprotic polar solvents such as dimethyl sulfoxide, sulfolane, N,N-dimethylformamide, N,N-dimethylacetamide, N,N-dimethylpropionamide, N-methylpyrrolidone, γ-butyrolactone, dimethyl carbonate, diethyl carbonate, ethylene carbonate, propylene carbonate, 1,3-dimethyl-2-imidazolidinone and 1,3-dimethyl-3,4,5,6-tetrahydro-2(1H)-pyridinone; nitrile solvents such as acetonitrile and propionitrile; and water. These solvents may be used alone, or a mixture thereof may be used. Ether solvents are preferable and, among these solvents, tetrahydrofuran is preferable. The use amount of the solvent is usually from 1 to 50 L, and preferably from 2 to 15 L, based on 1 kg of the compound.

The reaction temperature is preferably within a range from −30° C. to 70° C., and more preferably from 0° C. to 50° C. The reaction time varies depending on the reaction temperature, the use amount of a reaction reagent and the like, and is usually from 1 to 10 hours. Proceeding of the reaction can be confirmed by conventional means such as thin-layer chromatography, gas chromatography and high-performance liquid chromatography.

There is no particular limitation on the order of mixing of reaction reagents. However, mixing is preferably carried out in such an order that a base is added in a mixture of Compound (III-A) and the solvent and then an alkyl halocarbonate or a dialkyl carbonate is added.

Compound (IV-A) is contained in the mixture obtained after completion of the reaction, and may be subjected to deprotection as it is, as described hereinafter, or may be subjected after subjecting to conventional post-treatments such as filtration, extraction, washing with water. As a matter of course, Compound (IV-A) may be subjected after extracting by conventional isolation treatments such as distillation, crystallization, or may be subjected after purifying by conventional purification treatments such as recrystallization; extraction and purification; distillation; treatments of absorption to activated carbon, silica, alumina and the like; chromatography such as silica gel column chromatography. Also, Compound (IV-A) may be obtained as a salt of any acid such as hydrochloric acid, benzoic acid or tartaric acid

Examples of Compound (IV-A) include methyl 1-benzyl-trans-3-hydroxypiperidin-4-ylcarbamate, methyl 1-(1-phenylethyl)-trans-3-hydroxypiperidin-4-ylcarbamate, methyl 1-(1-phenylpropyl)-trans-3-hydroxypiperidin-4-ylcarbamate, methyl 1-(1-phenyl-2-methylpropyl)-trans-3-hydroxypiperidin-4-ylcarbamate, ethyl 1-benzyl-trans-3-hydroxypiperidin-4-ylcarbamate, ethyl 1-(1-phenylethyl)-trans-3-hydroxypiperidin-4-ylcarbamate, ethyl 1-(1-phenylpropyl)-trans-3-hydroxypiperidin-4-ylcarbamate, ethyl 1-(1-phenyl-2-methylpropyl)-trans-3-hydroxypiperidin-4-ylcarbamate, isopropyl 1-benzyl-trans-3-hydroxypiperidin-4-ylcarbamate, isopropyl 1-(1-phenylethyl)-trans-3-hydroxypiperidin-4-ylcarbamate, isopropyl 1-(1-phenylpropyl)-trans-3-hydroxypiperidin-4-ylcarbamate, isopropyl 1-(1-phenyl-2-methylpropyl)-trans-3-hydroxypiperidin-4-ylcarbamate, tert-butyl 1-benzyl-trans-3-hydroxypiperidin-4-ylcarbamate, tert-butyl 1-(1-phenylethyl)-trans-3-hydroxypiperidin-4-ylcarbamate, tert-butyl 1-(1-phenylpropyl)-trans-3-hydroxypiperidin-4-ylcarbamate, and tert-butyl 1-(1-phenyl-2-methylpropyl)-trans-3-hydroxypiperidin-4-ylcarbamate. Tert-butyl 1-benzyl-trans-3-hydroxypiperidin-4-ylcarbamate is preferable. When a racemic form is used as Compound (III-A), the obtained Compound (IV-A) is also usually a racemic form and, when an optically active form is used as Compound (III-A), the obtained Compound (IV-A) is also usually the optically active form.

The step of removing a substituent on a nitrogen atom constituting a piperidine ring from Compound (IV-A) is carried out under the condition, which is inert to carbamate, where an amino group protected with a benzyl type protecting group is made free. In this step, a hydrogenation reaction is usually carried out and, for example, a method for reacting Compound (IV-A) with hydrogen in the presence of palladium on carbon, a method for reacting Compound (IV-A) with hydrogen in the presence of palladium hydroxide, and a method for reacting Compound (IV-A) with sodium in the presence of liquid ammonia are used. Among these methods, a method for reacting Compound (IV-A) with hydrogen in the presence of palladium on carbon is preferable.

Palladium on carbon may be a wet product or a dry product. The content of a palladium atom is usually from 0.5 to 50% by weight, and preferably from 5 to 20% by weight. It is also possible to use, as palladium on carbon, commercially available palladium on carbon, and those prepared by any known method. The use amount of palladium on carbon is usually within a range from 0.1 to 50 g, and preferably from 1 to 20 g, in terms of the palladium atom based on 1 kg of Compound (IV-A). Palladium supported on carbon is usually zero-valent and when a di- or tetra-valent palladium compound is supported, it is preferably used after reducing to zero valence by a conventional method.

It is also possible to use, as hydrogen, a commercially available hydrogen gas, and those generated by any known method. The hydrogen pressure during the reaction is usually from 0.1 to 5 MPa, and preferably from 0.1 to 1 MPa. It is also possible to use as a mixed gas with an inert gas such as nitrogen or argon. In that case, the hydrogen pressure during the reaction is the same as the above hydrogen pressure.

The reaction of Compound (IV-A) with hydrogen is usually carried out in a solvent. The solvent which does not interfere with the reaction is available, and examples thereof include aliphatic hydrocarbon solvents such as pentane, hexane, isohexane, heptane, isoheptane, octane, isooctane, nonane, isononane, decane, isodecane, undecane, dodecane, cyclopentane, cyclohexane, methylcyclohexane, tert-butylcyclohexane and petroleum ether; ether solvents such as tetrahydrofuran, methyltetrahydrofuran, diethyl ether, dipropyl ether, diisopropyl ether, dibutyl ether, dipentyl ether, dihexyl ether, diheptyl ether, dioctyl ether, tert-butyl methyl ether, cyclopentyl methyl ether, 1,2-dimethoxyethane and diethylene glycol dimethyl ether; alcohol solvents such as methanol, ethanol, 1-propanol, 2-propanol, butyl alcohol, isobutyl alcohol, tert-butyl alcohol, 1-pentanol, 2-pentanol, isopentyl alcohol, 1-hexanol, 2-hexanol, isohexyl alcohol, 1-heptanol, 2-heptanol, 3-heptanol, isoheptyl alcohol, ethylene glycol monomethyl ether, ethylene glycol monoethyl ether, ethylene glycol monopropyl ether, ethylene glycol monoisopropyl ether, ethylene glycol monobutyl ether, ethylene glycol monoisobutyl ether, ethylene glycol mono-tert-butyl ether, diethylene glycol monomethyl ether, diethylene glycol monoethyl ether, diethylene glycol monopropyl ether, diethylene glycol monoisopropyl ether, diethylene glycol mono-n-butyl ether, diethylene glycol monoisobutyl ether and diethylene glycol mono-tert-butyl ether; ester solvents such as ethyl acetate, propyl acetate, isopropyl acetate, butyl acetate, isobutyl acetate, tert-butyl acetate, amyl acetate and isoamyl acetate; aprotic polar solvents such as dimethyl sulfoxide, sulfolane, N,N-dimethylformamide, N,N-dimethylacetamide, N,N-dimethylpropionamide, N-methylpyrrolidone, γ-butyrolactone, dimethyl carbonate, diethyl carbonate, ethylene carbonate, propylene carbonate, 1,3-dimethyl-2-imidazolidinone and 1,3-dimethyl-3,4,5,6-tetrahydro-2(1H)-pyridinone; and water. These solvents may be used alone, or may be used as a mixture thereof. Alcohol solvents are preferable and, among these alcohol solvents, ethanol is more preferable. The use amount of the solvent is usually from 1 to 50 L, and preferably from 2 to 15 L, based on 1 kg of the compound.

The reaction temperature is usually within a range from 0 to 100° C., and preferably from 20 to 70° C. The reaction time varies depending on the reaction temperature, the use amount of a reaction reagent, the hydrogen pressure and the like, and is usually from 1 to 24 hours. Proceeding of the reaction can be confirmed by conventional means such as thin-layer chromatography, gas chromatography and high-performance liquid chromatography.

There is no particular limitation on the order of mixing reaction reagents. For example, mixing can be carried out by a method for mixing Compound (IV-A) with palladium on carbon, if necessary, in the presence of a solvent and adding hydrogen to the obtained mixture, a method for adding Compound (IV-A) to palladium on carbon under a hydrogen atmosphere or the like. A method for mixing Compound (IV-A) with palladium on carbon in the presence of a solvent and adding hydrogen to the obtained mixture is preferable.

Compound (V-A) is contained in the mixture obtained after completion of the reaction and, for example, when such mixture is subjected to conventional post-treatments such as filtration, extraction, washing with water and then subjected to conventional isolation treatments such as distillation, crystallization, Compound (V-A) can be extracted. At this time, Compound (V-A) may be obtained as a salt of any acid such as hydrochloric acid, benzoic acid or tartaric acid. The thus isolated Compound (V-A) or a salt thereof can be further purified by conventional purification treatments such as recrystallization; extraction and purification; distillation; treatments of absorption to activated carbon, silica, alumina and the like; and chromatography such as silica gel column chromatography.

Examples of Compound (V-A) include methyl trans-3-hydroxypiperidin-4-ylcarbamate, ethyl trans-3-hydroxypiperidin-4-ylcarbamate, isopropyl trans-3-hydroxypiperidin-4-ylcarbamate, and tert-butyl trans-3-hydroxypiperidin-4-ylcarbamate. Tert-butyl trans-3-hydroxypiperidin-4-ylcarbamate is preferable. When a racemic form is used as Compound (IV-A), the obtained Compound (V-A) is also usually the racemic form and, when an optically active form is used as Compound (IV-A), the obtained Compound (V-A) is also usually the optically active form.

### EXAMPLES

The present invention will be described in more detail by way of Examples, but the present invention is not limited to these Examples.

### Reference Example 1

### Production of 1-benzyl-1,2,3,6-tetrahydropyridine

After mixing 10 g (126 mmol) of pyridine and 20 mL of toluene, 21.6 g (126 mmol) of benzyl bromide was added dropwise to the mixture while maintaining the inner temperature of the mixture at 20° C. After completion of the dropwise addition, the obtained mixture was stirred for 1 hour while heating in an oil bath at 110° C. The reaction mixture was cooled to about room temperature and 400 mL of ethanol was added thereto, followed by stirring and further divisional addition of 9.6 g (253 mmol) of sodium borohydride over 50 minutes. After completion of the addition, the obtained mixture was stirred at room temperature for 19.5 hours. To the reaction mixture, 200 mL of water was added, and then insolubles were removed by filtration. The insolubles were washed with ethyl acetate, and the filtrate and the wash were combined, and then 200 mL of ethyl acetate was added. However, the organic layer and the aqueous layer were not separated. Therefore, ethanol was distilled off from the mixture under reduced pressure to the extent that the obtained mixture is separated into the organic layer and the aqueous layer. Then, 300 mL of ethyl acetate was added to the obtained residue and the solution was extracted. The obtained organic layer was washed three times with 50 mL of saturated saline and then dried over anhydrous sodium sulfate. The solvent was distilled off under reduced pressure and the obtained residue was purified by silica gel column to obtain 15.5 g of 1-benzyl-1,2,3,6-tetrahydropyridine. Yield: 71%.

### Reference Example 2

### Production of 3-benzyl-7-oxa-3-aza-bicyclo[4.1.0]heptane

After mixing 5.0 g (28.9 mmol) of 1-benzyl-1,2,3,6-tetrahydropyridine obtained in Reference Example 1 and 7.5 mL of toluene, 50 mL of water was added to the obtained mixture and also 4.9 g (43.3 mmol) of trifluoroacetic acid was added dropwise. During the dropwise addition, the inner temperature of the mixture was from 24.7 to 28.2° C. After completion of the dropwise addition, the obtained mixture was stirred at room temperature for 0.5 hours. After stirring and further liquid separation, the aqueous layer was extracted and the organic layer was extracted with 5 mL of water. The obtained aqueous layers were combined and, after adjusting to 10° C., 9.3 g (52.0 mmol) of N-bromosuccinimide was divisionally added over 1 hour. During the addition, the inner temperature of the mixture was from 12.0 to 16.3° C. The obtained mixture was stirred at room temperature for 13 hours and then cooled to 4° C., and 23.0 g (145 mmol) of an aqueous 25% by weight sodium hydroxide solution was added dropwise. During the dropwise addition, the inner temperature of the mixture was from 4.4 to 9.0° C. After completion of the dropwise addition, the obtained mixture was stirred at room temperature for 3 hours and then 100 mL of toluene was added. The solution was extracted and the obtained organic layer was washed with 30 mL of saturated saline. The obtained organic layer was dried over sodium sulfate and then the solvent was distilled off under reduced pressure to obtain 3.9 g of 3-benzyl-7-oxa-3-aza-bicyclo[4.1.0]heptane. Yield: 71%.

The following compound numbers of Examples correspond to the numbers used for the following schemes.

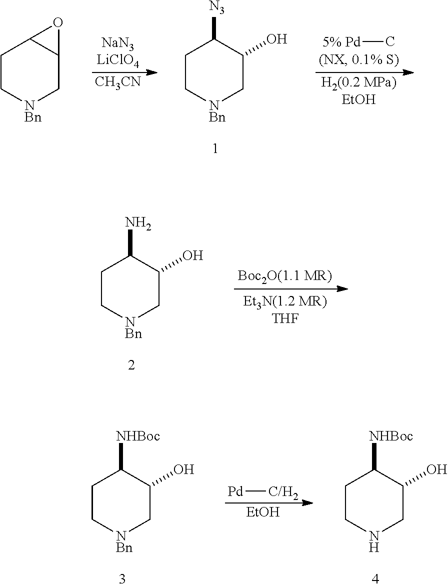

### Example 1

### Production of (3RS,4RS)-4-azido-1-benzylpiperidin-3-ol (Compound (1))

After mixing 0.76 g (4.0 mmol) of 3-benzyl-7-oxa-3-aza-bicyclo[4.1.0]heptane obtained in Reference Example 2 and 15 mL of acetonitrile, 0.85 g (8.0 mmol) of lithium perchlorate and 0.34 g (5.2 mmol) of sodium azide were added thereto. The obtained mixed solution was stirred at 55 to 65° C. for 5 hours. After completion of the reaction, 20 mL of water was added to the obtained mixture and then the solution was extracted twice with 20 mL of ethyl acetate. The obtained organic layers were combined, washed with 20 mL of saturated saline and then dried over anhydrous sodium sulfate. The obtained organic layer was partially concentrated to obtain 2.2 g of an ethyl acetate solution containing Compound (1). A portion of the obtained solution was concentrated and NMR of the residue was measured. As a result, a peak assigned to (3RS,4RS)-3-azido-1-benzylpiperidin-4-ol, i.e. an undesired position isomer was not recognized.

### Example 2

### Production of (3RS,4RS)-4-amino-1-benzylpiperidin-3-ol (Compound (2))

After mixing 2.2 g of an ethyl acetate solution containing Compound (1) obtained in Example 1 and 20 mL of ethanol in an autoclave reactor, and the atmosphere inside the system was replaced by a nitrogen atmosphere. After adding 184 mg of 5% by weight of palladium on carbon (50% by weight of a wet product, NX type, containing 0.1% by weight of sulfur, manufactured by N.E. CHEMCAT CORPORATION, Lot. 21A-040629) thereto, the atmosphere inside the system was replaced by hydrogen, followed by stirring under a hydrogen pressure of 0.1 to 0.2 MPa at room temperature for 2 hours. After completion of the reaction, the catalyst was removed by filtration and the obtained filtrate was concentrated to obtain 2.2 g of a mixture containing Compound (2). Without further purification, the entire amount of the mixture was used as it is in Example 3.

A portion of the obtained solution was concentrated and NMR of the residue was measured. As a result, a peak assigned to (3RS,4RS)-3-amino-1-benzylpiperidin-4-ol, i.e. an undesired position isomer was not recognized.

### Example 3

### Production of tert-butyl (3RS,4RS)-1-benzyl-3-hydroxypiperidin-4-ylcarbamate (Compound (3))

After mixing 2.2 g of Compound (2) obtained in Example 2 and 10 mL of tetrahydrofuran, the obtained mixture was ice-cooled and 0.67 mL of triethylamine and 1.0 mL of di-tert-butyl dicarbonate was added thereto, and then the obtained mixture was stirred at room temperature for 4 hours. Under ice cooling, 20 mL of water was added to the reaction mixture and then the solution was extracted twice with 20 mL of ethyl acetate. The obtained organic layers were combined, washed with saturated saline, dried over anhydrous sodium sulfate and then concentrated. The obtained residue was purified by silica gel column chromatography (n-heptane/ethyl acetate=1/1 to only ethyl acetate) to obtain 0.76 g of Compound (3). A total yield from Example 1 was 62% (in terms of 3-benzyl-7-oxa-3-aza-bicyclo[4.1.0]heptane).

### Example 4

### Production of tert-butyl (3RS,4RS)-3-hydroxypiperidin-4-ylcarbamate (Compound (4))

After mixing 0.76 g (2.5 mmol) of Compound (3) obtained in Example 3 and 10 mL of ethanol in an autoclave reactor, the atmosphere inside the system was replaced by a nitrogen atmosphere. After adding 0.15 g of 10% by weight of palladium on carbon (50% by weight of a wet product, PE type, manufactured by N.E. CHEMCAT CORPORATION, Lot. 217-013020) thereto, the atmosphere inside the system was replaced by hydrogen, followed by stirring under a hydrogen pressure of 0.4 to 0.6 MPa at 45 to 55° C. for 2 hours. After completion of the reaction, the catalyst was removed by filtration and the obtained filtrate was concentrated to obtain 0.49 g of Compound (4). Yield: 91%.

### Comparative Example 1

In the same manner as in Example 1, except that lithium perchlorate was not used in Example 1, the reaction was carried out. The reaction mixture was analyzed by thin-layer chromatography. As a result, the reaction scarcely proceeded.

### Comparative Example 2

In the same manner as in Example 1, except that the same molar amount of ethyl 7-oxa-3-aza-bicyclo[4.1.0]heptane-3-carboxylate was used in place of 3-benzyl-7-oxa-3-aza-bicyclo[4.1.0]heptane in Example 1, the reaction was carried out. The reaction mixture was concentrated and NMR of the residue was measured. As a result, a production ratio of ethyl trans-4-azido-3-hydroxypiperidine-1-carboxylate to ethyl trans-3-azido-4-hydroxypiperidine-1-carboxylate was about 1:1.

### INDUSTRIAL APPLICABILITY

An N-substituted-trans-4-azidopiperidin-3-ol obtained by the present invention is useful as various chemical products such as a pharmaceutical intermediate (see, for example, International Publication No. WO 2007/039462 or the like), the present invention is industrially applicable as its production method.

1. A method for producing an N-substituted-trans-4-azidopiperidin-3-ol represented by formula (II-1) 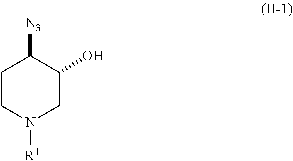
R¹ is as defined below, which comprises reacting an N-substituted-3,4-epoxypiperidine represented by formula (I): 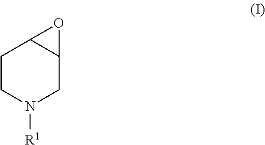
wherein R¹ represents an aralkyl group having 7 to 24 carbon atoms or an alkyl group having 1 to 12 carbon atoms, with sodium azide in the presence of an inorganic lithium salt.

2. The method according to claim 1, wherein the inorganic lithium salt is a lithium halide or a lithium perhalogenate.

3. The method according to claim 1, wherein the inorganic lithium salt is lithium chloride or lithium perchlorate.

4. The method according to claim 1, wherein the N-substituted-3,4-epoxypiperidine represented by the formula (I) is a compound represented by formula (I-A): 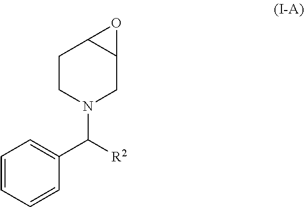
wherein R² represents an aralkyl group having 7 to 17 carbon atoms, an alkyl group having 1 to 11 carbon atoms, phenyl group or a hydrogen atom, and the N-substituted-trans-4-azidopiperidin-3-ol represented by formula (II-1) is an azide compound represented by formula (II-A): 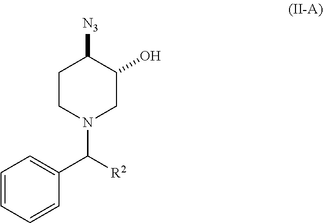
wherein R² is as defined above.

5. The method according to claim 4, wherein R² in formulas (I-A) and (II-A) is a hydrogen atom.

6. A method for producing an amino compound represented by formula (III-A): 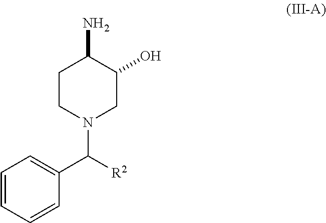
wherein R² is as defined below, which comprises reacting an N-substituted-3,4-epoxypiperidine represented by formula (I-A): 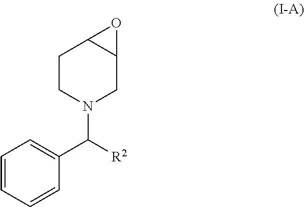
wherein R² represents an aralkyl group having 7 to 17 carbon atoms, an alkyl group having 1 to 11 carbon atoms, a phenyl group or a hydrogen atom, with sodium azide in the presence of an inorganic lithium salt; and reducing the obtained an azide compound represented by formula (II-A): 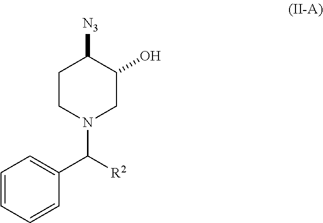
wherein R² is as defined above.

7. The method according to claim 6, wherein R² in formulas (I-A), (II-A) and (III-A) is a hydrogen atom.

8. The method according to claim 6, wherein reduction is the reaction of the azide compound represented by formula (II-A) with hydrogen in the presence of sulfur-containing palladium on carbon.

9. A method for producing a trans-4-alkoxycarbonylaminopiperidin-3-ol represented by formula (V-A): 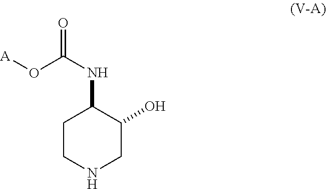
wherein A is as defined below, which comprises reacting an N-substituted-3,4-epoxypiperidine represented by formula (I-A): 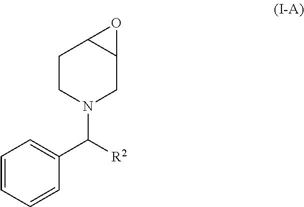
wherein R² represents an aralkyl group having 7 to 17 carbon atoms, an alkyl group having 1 to 11 carbon atoms, a phenyl group or a hydrogen atom, with sodium azide in the presence of an inorganic lithium salt; reducing the obtained azide compound represented by formula (II-A): 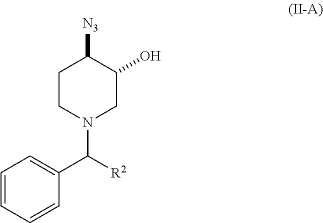
wherein R² is as defined above, to obtain an amino compound represented by formula (III-A): 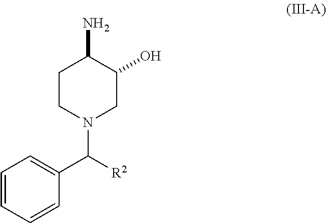
wherein R² is as defined above; protecting an amino group of the amino compound represented by the above formula (III-A) to obtain a carbamate compound represented by formula (IV-A): 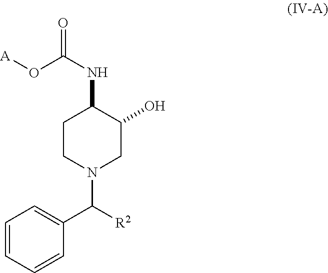
wherein R² has the same meaning as defined above, and A represents an alkyl group having 1 to 12 carbon atoms, and then removing a substituent on a nitrogen atom constituting a piperidine ring from the carbamate compound represented by the above formula (IV-A).

10. The method according to claim 9, wherein A in formulas (IV-A) and (V-A) is a tert-butyl group.
